(19) United States
(12) Patent Application Publication
(10) Pub. No.: US 2016/0008431 A1
(43) Pub. Date: Jan. 14, 2016

(54) Tumor Therapy With Antitumor Agents In Combination With Sindbis Virus-Based Vectors

(71) Applicant: New York University, New York, NY (US)

(72) Inventors: Daniel Meruelo, Scarborough, NY (US); Tomer Granot, Brooklyn, NY (US); Jen-Chieh Tseng, Canton, MA (US)

(21) Appl. No.: 14/798,077

(22) Filed: Jul. 13, 2015

Related U.S. Application Data
(63) Continuation of application No. 13/133,680, filed on Sep. 19, 2011, now abandoned.
(60) Provisional application No. 61/138,944, filed on Dec. 18, 2008.

(51) Int. Cl. A61K 38/18; A61K 31/337

(52) U.S. Cl. CPC A61K 38/1866; A61K 31/337; C12N 2770/36132; C12N 2770/36145

(57) ABSTRACT

A method for treating malignant tumors with Sindbis viral based vectors in combination with antitumor agents and pharmaceutical formulations for use in such treatment.

### CROSS REFERENCE TO RELATED APPLICATIONS

The present application claims the benefit of U.S. Provisional Patent Application Ser. No. 61/138,944, filed Dec. 18, 2008, which is hereby incorporated by reference in its entirety.

The United States Government has certain rights to this invention by virtue of funding received from U.S. Public Health Service Grant No. CA 100687, from the National Cancer Institute, National Institutes of Health, Department of Health and Human Services.

### FIELD OF THE INVENTION

This present invention is directed to the treatment of tumors in mammals using antitumor agents in combination with Sindbis virus based vectors.

### BACKGROUND OF THE INVENTION

CPT-11 (irinotecan), a topoisomerase 1 inhibitor, is a clinically approved first-line anti-cancer agent that has been used for a number of cancer types, including colorectal cancer and ovarian cancer (1, 2). However, CPT-11 by itself is often not sufficient to cure the disease. Furthermore, some tumors have been shown to be resistant to CPT-11 therapy (3). Similarly, Taxol® (Paclitaxel), a microtubule-stabilizing agent, has been used as a chemotherapeutic agent for a number of tumor types (4). Another therapeutic approach has been the use of oncolytic viruses or viral-vectors, such as Sindbis virus-based vectors, to target tumors in vivo (5). Sindbis virus vectors are believed to target tumor cells because the receptor for the virus—the high-affinity laminin receptor (LAMR)—is up-regulated in various tumors (6). Yet other factors, which might enhance the vectors' ability to replicate in tumor cells, may also be involved. Although Sindbis-based vectors have been shown to specifically target tumor cells in mice, and to induce apoptosis in these cells, the vectors alone are often not sufficient to cure the disease (7).

Chemotherapeutic agents have long been used as a first-line treatment for a variety of cancers. More recently, viral-vector based treatments have also been shown to have an antitumor effect in mouse tumor models. However, some tumors appear to be resistant or partially resistant to these treatments. Novel approaches are therefore needed to maximize the therapeutic potential of these treatments. Combinatorial therapy is the use of several treatments simultaneously. This approach has been shown to he effective in various tumor models. Two distinct treatments can result in enhancement of one or both therapies or in synergism between the therapies (8,9). It is known, for example, that chemotherapy can affect the behavior of tumor cells by altering the expression of various genes. It is not known, however, whether or not these changes can affect the susceptibility of these tumor cells to infection with viruses or viral vectors. Similarly, it is not known if anti-cancer treatment with viruses or viral vectors can affect the susceptibility of tumor cells to chemotherapeutic agents.

U.S. Pat. No. 7,306,712 discloses that vectors based on Sindbis virus, a blood-borne alphavirus transmitted through mosquito bites, infect tumor cells specifically and systematically throughout the body. The tumor specificity of Sindbis vectors is mediated by the 67-kDa high-affinity laminin receptor (LAMR), which is over-expressed in several types of human tumors and has the advantageous property that, without carrying cytotoxic genes, induce apoptosis in mammalian cells. Furthermore, as Sindbis vectors are capable of expressing very high levels of their transduced genes in infected tumor cells, they can be advantageously used With suicide genes, whereby the efficient production of the enzymes required for sufficient prodrug conversion and use of said genes is ensured.

Co-pending U.S. patent application Ser. No. 10/920,030 discloses methods and compositions for detecting cancer cells and monitoring cancer therapy using replication defective Sindbis virus vectors.

U.S. Pat. No. 7,303,798 discloses novel defective Sindbis virus vectors and their use in treating tumors in mammals.

Co-pending U.S. Patent Application Ser. No. 60/030,362 discloses replication competent Sindbis virus vectors and there use in treating tumors in mammals.

### SUMMARY OF THE INVENTION

Presented herein are experiments which demonstrate the therapeutic value of combining known antitumor agents with Sindbis virus-based vector treatment. The data presented herein shows that treatment with chemotherapeutic agents or with Sindbis virus vectors alone can extend the survival of tumor-bearing mice by a few weeks. However, when the two treatments were combined, the survival was prolonged for much longer periods of time. Surprisingly, a significant proportion of the doubly treated mice remained tumor-free and appeared healthy for over 200 days. This is a significant proportion of their life span. These results indicate that combining antitumor agents with Sindbis virus vector treatment is an effective method for treating some types of cancer.

To study the potential benefit of combining antitumor agents and viral-based treatments, a well-established, aggressive in vivo tumor model was tested using the clinically approved chemotherapeutic agent CPT-11, together with Sindbis virus vector treatment. The tumor model that was chosen was ES2/Fluc cells, a human ovarian cancer model. ES2/Fluc cells express the firefly luciferase gene so that the tumor growth can be monitored by longitudinal imaging the mice. ES2 cells have been shown to be resistant to certain drugs (10). In addition, in vivo experiments in SCID mice have shown that unmodified, defective Sindbis virus vectors (Sindbis/LacZ) per se can only prolong the survival of ES2/Fluc tumor-bearing mice by 1-2 weeks. Payloads can enhance the efficacy of the vectors, and vectors carrying therapeutic genes like Sindbis/IL-12 can prolong the survival of the tumor-bearing mice by several more weeks (7); however, complete tumor remission has not been previously observed in this aggressive tumor model using any viral vector or antitumor agent.

In another embodiment, evidence is presented that shows that modulating tumor vascular leakiness, using Sindbis virus vectors carrying the VEGF gene and/or metronomic chemotherapy regimens significantly enhances tumor vascular permeability and directly enhances oncolytic Sindbis vector targeting. Since host-derived vascular endothelium cells are genetically stable and less likely to develop resistance to chemotherapeutics, a combined metronomic chemotherapeutics and oncolytic viruses regimen provides a new approach for cancer therapy.

### BRIEF DESCRIPTION OF THE DRAWINGS

FIGS. 1A and 1B: Survival of tumor-bearing mice treated with Sindbis+CPT-11. (A) Mice were injected with 1.5×10⁶ ES2/Fluc cells on day 0. Mice were then divided into 4 groups of 10. Treatment started on day 5, and lasted for 5 weeks, in which mice were treated 4 times a week. Mice treated with Sindbis/LacZ received I.P. injection of ˜10⁷ plaque-forming units of the vector in 0.5 mL OptiMEM; mice treated with CPT-11 received I.P. injections of 15 mg/kg body weight of the recipient of CPT-11 in 250 mL PBS. Mice treated with the combined treatments were treated with Sindbis/LacZ and CPT-11 on the same day (Sindbis/LacZ in the morning, and CPT-11 in the evening). The results showed that mice survival was slightly prolonged with the single treatments, but was substantially prolonged with the combined treatment. The experiment was repeated twice (for a total of 20 mice per group), and the results were combined into one graph. (B) Tumor load in mice treated with Sindbis/LacZ+CPT-11. On day 4 after injecting the mice with 10 million ES2/Fluc cells, tumors developed in various IP locations (top). As mentioned above, in the first experiment, 6 out of 10 of the mice treated with Sindbis/LacZ+CPT-11 survived, and appeared to be cured of the cancer. These mice were imaged on day 154 after injecting the cancer cells, and 3 mice were shown to be completely tumor free (bottom; mice 3, 5, and 6). Three other mice (1, 2, and 4) showed a small amount of residual luminescent cells, but these cells did not appear to be growing (data not shown), and did not seem to have a serious effect on the morbidity of the mice, which appear to be healthy. The day 4 and day 154 images were taken under identical conditions (High resolution binning; field of view=D; 15 seconds exposure; color scale=50-1000).

FIG. 2A-2D: Survival and tumor load in tumor-bearing mice treated with Sindbis+Taxol®. Mice were injected with 4×10⁶ ES2/Fluc cells on day 0. Mice were then divided into 4 groups of 9. Mice were treated with Sindbis/LacZ (I.P.) daily from day 1 to day 11, and with Taxol® (0.4 mg/mouse) on day 3, 6 and 10. (B) Quantitative analysis of tumor growth, day 1 tumor load signal was set at 100% for each individual mouse for comparison with later images (day 3, 6, and 10). (C) Mouse survival was monitored. (D) The surviving mice were imaged on day 46 to determine if they have any tumors.

FIG. 3 is a graph showing the effect of Sindbis virus vectors with and without CPT-11 treatment on pancreatic cancer in Mia Paca mice, a model for pancreatic cancer.

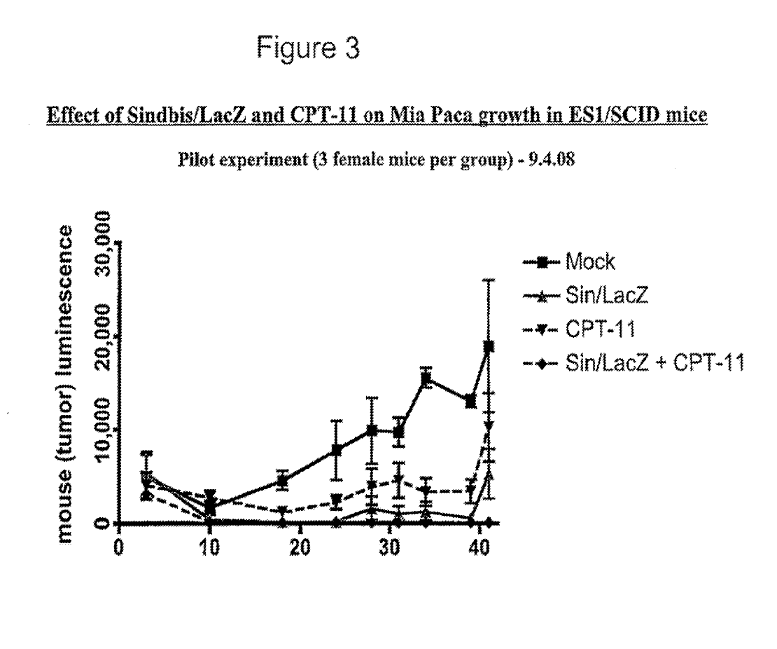

FIG. 4A-4C. Dual fluorescent imaging of tumor vasculature and its leakiness. In SCID mice carrying s.c. BHK tumors, both Qtracker® and AngioSense® detect tumor vasculature 100 min after i.v. injection of a 200 μL mixture of both Qtracker® (0.1 μM) and AngioSense® (3.3 μM). However, the AngioSense® can visualize tumor vascular leakiness after 24 hours of probe injection. A, raw image data after sequential acquiring of the indicated excitation/emission matrix. B, the unmixed concentration maps for Qtracker® and AngioSense®. C, the composite images of Qtracker (green) and AngioSense (red) signals.

FIG. 5A-5C. Sindbis viral vector transduces cancer cells via tumor vascular leakiness. A, kinetic images of SCID/BHK s.c. tumors after i.v. injection of AngioSense® (0.66 nmol) and RD-Sindbis/mPlum (˜10⁷ particles) on day 0. B, reconstructed concentration maps for mPlum and AngioSense® of the day 3 images. The mPlum signals are well associated with dead tumor tissue that shows little AngioSense® signals. C, using a RD-Sindbis/Fluc vector that carries a firefly luciferase, instead of a mPlum gene, enable detection of vector infection and its correlation with vascular leakiness as early as day 1.

FIG. 6A-6D. VEGF enhances tumor vascular leakiness and promotes Sindbis vector targeting. RD-Sindbis/VEGF vector (˜10⁷ particles/mL) were mixed with RD-Sindbis/Fluc vector (˜10⁷ particles/mL) at 1:1 ratio. A mixture of RD-Sindbis/LacZ and RD-Sindbis/Fluc was used as a control. On day 0 and 1, vector mixture (500 μL) was i.p. injected into SCID mice bearing s.c. BHK tumors. AngioSense® 750 (0.66 nmol) was i.v. injected on day 1. A, AngioSense® signal on day 3 shows enhancement of vascular permeability in the tumors of mice receiving the mixture containing RD-Sindbis/VEGF vectors. B, tumor AngioSense® signals in total fluorescent efficiency on day 1 (100 min after probe injection), 2, 3, 4 and 7. C, bioluminescent imaging of luciferase activities indicates VEGF promotes vector delivery and transduction. D, quantitative presentation of luciferase activities in tumors.

FIG. 7A-7D. Paclitaxel causes enhancement in tumor vascular leakiness and synergizes with oncolytic Sindbis vector in cancer therapy. On day 0, treatments of 1:1 mixture of RD-Sindbis/VEGF:RC-Sindbis/Fluc (0.5 mL, each has ˜10⁷ particle/mL) were injected into tumor-bearing mice via the tail veins. We used 1:1 RD-Sindbis/LacZ:RC-Sindbis/Fluc mixture as a control. AngioSense® 750 (0.66 nmol) was i.v. injected on day 1. A, i.p. Paclitaxel treatments (Taxol®, 16 mg/Kg or 48 mg/m² on day 2, 3 and 6, compared with maximum tolerated dose of 175 mg/m² in human) cause vascular insults and enhance tumor vascular leakiness. The enhanced vascular leakiness further synergizes with RD-Sindbis/VEGF and promotes oncolytic replication of RC-Sindbis/Fluc vector in s.c. N2a tumors. B, quantitative presentation of luciferase signals in tumors. C and D, quantitative presentation of AngioSense® signals of tumors receiving indicated treatments.

FIG. 8A-B. The combined treatments enhance the efficacy of Sindbis viral vectors. A, relative growth curves of s.c. N2a tumors treated with Paclitaxel alone (Taxol®) or untreated (Ctrl). B, relative tumor growth curves of different treatment groups as in FIG. 5.

FIG. 9A-9C. Cisplatin causes enhancement in tumor vascular leakiness and synergizes with oncolytic Sindbis vector in cancer therapy. Starting on day 0, daily treatments of Cisplatin (4 mg/Kg or 12 mg/m² compared with maximum tolerated dose of 100 mg/m² in humans) were i.p. injected into SCID mice bearing s.c. N2a tumors. One last Cisplatin treatment was administrated on day 4. A, to visualize vascular leakiness, AngioSense® 750 (0.66 nmol) was i.v. injected on day 1 and imaged on day 2. B, RC-Sindbis/Fluc was i.v. injected on day 0 and day 2. Luciferase activities in tumors that indicated active vector propagation were monitored on day 3. C, relative tumor growth curves of different treatment groups.

### DETAILED DESCRIPTION OF THE INVENTION

In one embodiment, the instant invention takes advantage of the affinity of Sindbis virus vectors for tumor cells, in particular, for solid tumors that express higher levels of high affinity laminin receptors, as compared to normal cells of the same lineage. The term “high affinity laminin receptor” or “LAMR” has its ordinary meaning in the art, i.e., the Mr 67,000 laminin receptor that can function as the receptor for Sindbis virus entry into cells (Wang et at, J. Virol. 1992, 66:4992-5001; Strauss et al., Arch. Virol. Suppl. 1994, 9:473-84).

Accordingly, the present invention provides a method for treating a mammal (e.g., human) suffering from a tumor that expresses greater levels of LAMR compared to normal cells of the same lineage. The method comprises administering to a mammal harboring such a tumor an amount of (a) a Sindbis virus vector and (b) an antitumor agent, wherein the amounts of (a) and (b) in combination are effective to treat the tumor and the vector has a preferential affinity for LAMR.

While not wishing to be bound by any particular theory, three sets of observations may account for the remarkable antitumor efficiency of Sindbis vector-based therapy of the present invention. First, the LAMR can function as the receptor for Sindbis virus entry into cells of most species (Wang et al., J. Virol., 1992, 66:4992-5001; and Strauss et al. Arch. Virol. Suppl., 1994, 9:473-484). Second, it is widely recognized that expression of the LAMR is markedly elevated in many types of cancers (Menard et al., Breast Cancer Res. Treat, 1998, 52:137-145). In fact, a significant correlation has been established between the increased expression of Mr 67,000 LAMR and cancers of the breast (Menard et al., 1998, supra; Paolo Viacava et al., J. Pathol., 1997, 182:36-44; Martignone et al., J. Natl. Cancer Inst., 1993, 85:398-402), thyroid (Basolo et al., Clin. Cancer Res., 1996, 2:1777-1780), colon (San Juan et al., J Pathol., 1996, 179:376-380), prostate (Menard S et al., Breast Cancer Res. Treat, 1998,52: 137-149, stomach (de Manzoni et al., Jpn J Clin. Oncol., 1998, 28:534-537), pancreas (Pelosi et al., J Pathol., 1997, 183:62-69), ovary (Menard et al., Breast Cancer Res. Treat, 1998, 52:137-145; and van den Brule et al., Eur J Cancer, 1996, 32A:1598-1602.), melanocytes (Taraboletti et al., J Natl. Cancer Inst., 1993, 85:235-240), lung (Menard et al., Breast Cancer Res. Treat, 1998, 52:137-145), liver (Ozaki et al., Gut, 1998, 43:837-842), endometrium, and uterus (van den Brule et al., Hum Pathol, 1996, 27:1185-1191). Indeed, data on more than 4000 cases of different tumors from diverse organs studied by immunohistochemistry are all concordant with a role for HALR in invasiveness, metastasis, and tumor growth (Menard et al., Breast Cancer Res. Treat., 1998, 52:137-145).

The vectors of the present invention do not infect normal cells to the same extent in vivo compared to tumor cells. This allows for a differential effect in vector therapy, e.g., infection by the vectors disclosed herein results in the death of tumor cells leading to tumor elimination without apparent deleterious effects to other tissues and organs of the treated subjects. This phenomenon may be explained by the observation that an increased number of LAMR in tumors versus normal cells leads to a high number of exposed or unoccupied receptors on tumor cells (Liotta, L. A. Cancer Research, 1986, 46:1-7; Aznavoorian et al., 1992, Molecular Aspects of Tumor Cell Invasion and Metastasis, pp. 1368-1383). For example, it has been demonstrated that breast carcinoma and colon carcinoma tissues contain a higher number of exposed (unoccupied) LAMR compared to benign lesions (Liotta et al., 1985, Exp. Cell Res., 156:117-26; Barsky et al., Breast Cancer Res. Treat., 1984, 4:181-188; Terranova et al., Proc. Natl. Acad. Sci. USA, 1983, 80:444-448). These excess unoccupied LAMR receptors on tumor cells, which are not found in normal cells, may be available for vector binding, infection, and induction of cell death.

In one embodiment, the invention advantageously provides a method for treating a mammal suffering from a tumor, in which the cells of the tumor express greater levels of LAMR compared to normal cells of the same lineage. The different levels of LAMRs result in target-mediated delivery, i.e., preferential binding of vectors of the invention to tumor cells. “Greater levels” of expression generally refer herein to levels that are expressed by tumor cells (as compared to non-tumor cells) and result in such preferential binding, e.g., at least a 3-fold greater binding, preferably at least a 30-fold greater binding and, most preferably at least a 300-fold greater binding. The increased level of expression in tumor cells can be evaluated on an absolute scale, i.e., relative to any other LAMR expressing non-tumor cells described, or on a relative scale, i.e., relative to the level expressed by untransformed cells in the same lineage as the transformed cancer cells (e.g., melanocytes in the case of melanoma; hepatocytes in the case of hepatic carcinoma; ovarian endothelial cells in the case of ovarian adenocarcinoma, renal endothelial or epithelial cells in the case of renal carcinoma).

As used herein, the term “tumor” refers to a malignant tissue comprising transformed cells that grow uncontrollably. Tumors include leukemias, lymphomas, myelomas, plasmacytomas, and the like; and solid tumors. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, epidermoid carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, neuroglioma, and retinoblastoma. As noted above, the method of the invention depends on expression of LAMRs by cells of the tumor targeted for treatment.

The term “about” or “approximately” usually means within an acceptable error range for the type of value and method of measurement. For example, it can mean within 20%, more preferably within 10%, and most preferably still within 5% of a given value or range. Alternatively, especially in biological systems, the term “about” means within about a log (i.e., an order of magnitude) preferably within a factor of two of a given value.

The term “therapeutically effective” when applied to a dose or an amount refers to that quantity of a compound or pharmaceutical composition that is sufficient to result in a desired activity upon administration to a mammal in need thereof. As used herein with respect to viral vectors or antitumor agents of the invention, the term “therapeutically effective amount/dose” refers to the amount/dose of a vector or pharmaceutical composition containing the antiviral agent that in combination is sufficient to produce an effective antitumor response upon administration to a mammal.

The preferred route of administration of the vectors of the present invention for treatment is parenteral and most preferably systemic. This includes, but is not limited to intravenous, intraperitoneal, intra-arteriole, intra-muscular, intradermal, subcutaneous, intranasal and oral. These routes of administration will permit homing of the vector to tumor cells. Therefore, Sindbis virus-based vectors disclosed herein have an advantage over other viral vectors that are not adapted to travel in the bloodstream. This property is largely responsible for the observation that systemic administration of Sindbis viral vectors by i.p. or i.v. injections, target and infect only tumors expressing greater amounts of LAMR than normal cells of the same lineage growing s.c., i.p., intrapancreatically, or in the lungs or in other organs such as the liver, the pancreas, the brain, etc. In terms of tumors of the brain, such as glioblostoma, Sindbis based vectors are particularly well-suited to treat such malignancies because the vectors cross the blood brain barrier.

Non-limiting Examples of Sindbis virus-based vectors for use in the present invention include defective Sindbis virus vectors disclosed in U.S. Pat. No. 7,303,998 and Ser. Nos. 11/876,522, 11/877,018 and 12/123,790, and replication competent Sindbis virus vectors disclosed in Ser. No. 60/030,367.

Both replication competent and replication defective Sindbis virus vectors can be used in the embodiments of the present invention. The therapeutically effective amounts of these vectors broadly ranges between about 10⁶ and about 10¹² per treatment. The vectors can also contain payloads of antitumor genes as described in U.S. Pat. No. 7,306,792. In one embodiment, the Sindbis virus vector is replication competent and the payload comprises a suicide gene, such as thymidine kinase as disclosed in Ser. No. 60/030,367.

In another embodiment, the present invention provides a method to treat any tumor in a mammal which expresses a unique cell surface antigen or tumor specific target. In this embodiment, the Sindbis virus vector contains a chimeric envelope protein comprising a LAMR binding domain of the Sindbis virus E2 protein and the Fc binding domain of Staphoccal Protein A. These vectors are used in conjunction with antibodies directed against a tumor specific target. This allows for tumor specific targeting of the Sindbis virus vector to any tumor cell containing the tumor specific target as defined herein. These vectors are disclosed in U.S. Pat. No. 6,436,998. “Tumor-specific target” is used herein to broadly define any molecule on the surface of a tumor cell, which can be used for selective or preferential targeting of this cell by the vectors of the invention. Tumor-specific cellular determinants for the vectors of the instant invention include without limitation any tumor cell surface protein, peptide, oligonucleotide, lipid, polysaccharide, and a small molecule ligand. Preferred tumor-specific cellular determinants of the invention are tumor-specific membrane proteins such as ErbB receptors, Melan A [MART1], gp100, tyrosinase, TRP-1/gp 75, and TRP-2 (in melanoma); MAGE-1 and MAGE-3 (in bladder, head and neck, and non-small cell carcinoma); HPV EG and E7 proteins (in cervical cancer); Mucin [MUC-1] (in breast, pancreas, colon, and prostate cancers); prostate-specific antigen [PSA] (in prostate cancer); carcinoembryonic antigen [CEA] (in colon, breast, and gastrointestinal cancers), LH/CG receptor (in choriocarcinoma), and such shared tumor-specific antigens as MAGE-2, MAGE-4, MAGE-6, MAGE-10, MAGE-12, BAGE-1, CAGE-1,2,8, CAGE-3 to 7, LAGE-1, NY-ESO-1/LAGE-2, NA-88; GnTV, and TRP2-INT2, etc. HALRs as well as other determinants (e.g., EGF receptors or .alpha..sub.v.beta..sub.3 integrins), which are expressed at higher levels on the surface of certain tumor cells, as compared to normal cells of the same lineage, are also encompassed by the term “tumor-specific target.”

Non-limiting examples of antitumor agents for use in the present invention are presented below in Table 1.

| | Agents | Cancer treatment |
|---|---|---|
| Alkylating | | |
| group | | |
| Nitrogen | Uramustine | non-Hodgkin lymphoma |
| mustards | Chlorambucil | Chronic lymphocytic leukemia, non-Hodgkin |
| analogues | Chlormethine | Hodgkin's lymphoma. (It has been derivatized into the |
| | | estrogen analogue estramustine, used to treat prostate |
| | | cancer) |
| | Cyclophosphamide | The main use of cyclophosphamide is together with |
| | | other chemotherapy agents in the treatment of |
| | | lymphomas, some forms of leukemia and some solid |
| | | tumors breast and ovarian cancer |
| | Ifosfamide | Testicular, breast cancer, Lymphoma (Non- |
| | | Hodgkin), Soft tissue sarcoma, Osteogenic sarcoma, |
| | | lung, cervical, bone, ovarial cancer |
| | Melphalan | multiple myeloma and ovarian cancer, and occasionally |
| | | malignant melanoma |
| | Bendamustine | leukemia, sarcoma |
| | Trofosfamide | undergoing clinical trials |
| Nitrosoureas | Carmustine | brain cancer (including glioma, glioblastoma |
| | | multiforme, medulloblastoma and astrocytoma), |
| | | multiple myeloma and lymphoma (Hodgkin's and non- |
| | | Hodgkin) |
| | Fotemustine | melanoma |
| | Lomustine | brain tumors |
| | Nimustine | brain tumors |
| | Prednimustine | chronic lymphocytic leukemia non-Hodgkin's |
| | | lymphomas, and other malignant conditions including |
| | | breast cancer. |
| | Ranimustine | chronic myelogenous leukemia and polycythemia vera |
| | Semustine | brain tumors, lymphomas, colorectal cancer, and |
| | | stomach cancer. |
| | Streptozocin | cancer of the pancreatic islet cells |
| Platinum | Carboplatin | ovarian carcinoma, lung, head and neck cancers |
| (alkylating-like) | Cisplatin | Small cell lung cancer, ovarian cancer lymphomas and |
| | | germ cell tumors |
| | Nedaplatin | head and neck cancer, small cell lung cancer, testicular |
| | | tumor |
| | Oxaliplatin | In combination with Fluorouracil and leucovorin known |
| | | as FOLFOX for treatment of colorectal cancer |
| | Triplatin tetranitrate | undergoing clinical trials |
| | Satraplatin | advanced prostate cancer |
| Alkylsulfonates | Busulfan | Chronic myelogenous (or myeloid) leukemia |
| | Mannosulfan | undergoing clinical trials |
| | Treosulfan | ovarian cancer |
| Hydrazines | Procarbazine | Hodgkin's lymphoma and certain brain cancers (such |
| | | as Glioblastoma multiforme) |
| Triazenes | Dacarbazine | malignant melanoma. Hodgkin lymphoma, sarcoma, |
| | | and islet cell carcinoma of the pancreas |
| | Temozolomide | refractory anaplastic astrocytoma |
| Anti- | | |
| metabolites | | |
| Group | | |
| Folic acid | Aminopterin | leukemia |
| | Methotrexate | Acute lymphoblastic leukemia |
| | Pemetrexed | pleural mesothelioma, non-small cell lung cancer |
| | Raltitrexed | colorectal cancer |
| Purine | Cladribine | Hairy cell leukemia |
| | Clofarabine | Acute lymphoblastic leukemia, Acute myeloid leukemia |
| | Fludarabine | chronic lymphocytic leukemia |
| | Mercaptopurine | Leukemia, pediatric non-Hodgkin's lymphoma |
| | Pentostatin | Hairy cell leukemia |
| | Tioguanine | acute leukaemias and chronic myeloid leukaemia. |
| Pyrimidine | Cytarabine | leukemia and non-Hodgkin lymphoma. |
| | Decitabine | chronic myeloid leukaemia. |
| | Fluorouracil (5-FU) | colorectal and pancreatic cancer |
| | Capecitabine | metastatic breast and colorectal cancers |
| | Floxuridine | colorectal cancer |
| | Gemcitabine | non-small cell lung cancer, pancreatic cancer, bladder cancer and |
| | | breast cance |
| Plant alkaloids and | | |
| Terpenoids group | | |
| Vica alkaloids and | Vincrisine | Its main uses are in non-Hodgkin's lymphoma |
| analogues | | as part of the chemotherapy regimen CHOP, |
| | | Hodgkin's lymphoma as part of MOPP or |
| | | COPP, or the less popular Stanford V |
| | | chemotherapy regimen, in acute lymphoblastic |
| | | leukemia, and in treatment for nephroblastoma |
| | | (Wilms tumor, a kidney tumor common in |
| | | children). |
| | Vinblastine | Hodgkin's lymphoma, non-small cell lung |
| | | cancer, breast cancer and testicular cancer. |
| | Vinorelbine | breast cancer and non-small cell lung cancer |
| | Vindesine | leukaemia, lymphoma, melanoma, breast |
| | | cancer, and lung cancer |
| | Vinflunine | bladder cancer |
| Podophyllotoxin | Etoposide | Ewing's sarcoma, lung cancer, testicular |
| | | cancer, lymphoma, non-lymphocytic leukemia, |
| | | and glioblastoma multiforme. |
| | Teniposide | childhood acute lymphocytic leukemia |
| Texanes | Paclitaxel | lung, ovarian, breast cancer, head and neck |
| | | cancer, and advance forms of Kaposi's |
| | | sarcoma. |
| | Docetaxel | breast, ovarian, and non-small cell lung cancer |
| Other | Trabectedin | breast, prostate, and paediatric sarcomas |
| Anti-tumour | | |
| antibiotics | | |
| group | | |
| Anthracycline | Doxorubicin | leukemias, Hodgkin's lymphoma, as well as cancers of |
| and related | | the bladder, breast, stomach, lung, ovaries, thyroid, soft |
| substances | | tissue sarcoma, multiple myeloma, |
| | Daunorubicin | leukaemia (acute myeloid leukemia and acute |
| | | lymphocytic leukemia |
| | Epirubicin | breast and ovarian cancer, gastric cancer, lung cancer, |
| | | and lymphomas. |
| | Aclarubicin | Acute leukaemia |
| | Zorubicin | breast cancer |
| | Idarubicin | acute lymphocytic leukemia |
| | Mitoxantrone | metastatic breast cancer, acute myeloid leukemia, and |
| | | non-Hodgkin's lymphoma. |
| | Valrubicin | bladder cancer |
| Cytotoxic | Bleomycin | Hodgkin lymphoma (as a component of the ABVD |
| antibiotics | | regimen), squamous cell carcinomas, and testicular |
| | | cancer, pleurodesis as well as plantar warts |
| | Plicamycin | testicular cancer |
| | mitomycins | upper gastro-intestinal (e.g. esophageal carcinoma) and |
| | | breast cancers, as well as by bladder instillation for |
| | | superficial bladder tumours. |
| Actinomycines | Actinomycin | gestational trophoblastic |
| | | neoplasia, rhabdomyosarcoma, Wilms' tumor |
| Topoisomerase | | |
| inhibitors group | | |
| Type I | Irinotecan | Its main use is in colon cancer, particularly in |
| | | combination with other chemotherapy agents. |
| | | This includes the regimen FOLFIRI which |
| | | consists of infusional 5-fluorouracil, leucovorin, |
| | | and irinotecan. |
| | Topotecan | ovarian cancer and lung cancer |
| Type II | Amsacrine | leukemia |
| | Etoposide phosphate | Ewing's sarcoma, lung cancer, testicular |
| | | cancer, lymphoma, non-lymphocytic leukemia, |
| | | and glioblastoma multiforme |
| | Teniposide | childhood acute lymphocytic leukemia |
| Monoclonal | | |
| antibodies group | | |
| Receptor tyrosine | Cetuximab | metastatic colorectal cancer and head |
| kinase | | and neck cancer |
| | Panitumumab | treatment of EGFR-expressing |
| | | metastatic colorectal cancer with disease |
| | | progression |
| | Trastuzumab | Breast cancer over-express erbB2 |
| | | receptor |
| CD20 | Rituximab | B cell non-Hodgkin's lymphoma, B cell |
| | | leukemia, and some autoimmune |
| | | disorders |
| | Tositumomab | treatment in patients with relapsed or |
| | | chemotherapy/rituxan refractory follicular |
| | | lymphoma. |
| Other | Alemtuzumab | chronic lymphocytic leukemia (CLL) and |
| | | T-cell lymphoma. |
| | Bevacizumab | colon cancer, breast cancer and non- |
| | | small cell lung cancer[ |
| | Edrecolomab | colon cancer |
| | Gemtuzumab ozogamicin | acute myelogenous leukemia. |
| Group | | |
| Protein | Imatinib | chronic myelogenous leukemia (CML), |
| kinase | | gastrointestinal stromal tumors (GISTs) and a |
| inhibitors | | number of other malignancies |
| | Gefitinib | locally advanced or metastatic non-small cell lung |
| | | cancer and cancers where EGFR overexpression is |
| | | involved |
| | Erlotinib hydrochloride | non-small cell lung cancer, pancreatic cancer and |
| | | several other types of cancer |
| | Sunitinib | renal cell carcinoma (RCC) and imatinib-resistant |
| | | gastrointestinal stromal tumor |
| | Sorafenib | primary kidney cancer (advanced renal cell |
| | | carcinoma) and advanced primary liver cancer |
| | | (hepatocellular carcinoma). |
| | Dasatinib | chronic myelogenous leukemia (CML), acute |
| | | lymphoblastic leukemia, metastatic melanoma. |
| Horomal | Dexamethasone | Hematological malignancies, Multiple myeloma |
| therapy | Finasteride | prostate cancer |
| | Aromatase inhibitors | breast cancer and ovarian cancer |
| | Tamoxifen | breast cancer |
| | Goserelin | prostate cancer |
| Other | Asparaginase | acute lymphoblastic leukemia |
| | Altretamine | refractory ovarian cancer |
| | Hydroxyurea | hematological malignancies, specifically |
| | | polycythemia vera and essential |
| | | thrombocytosis |
| | Pentostatin | hairy cell leukemia |
| | Estramustine | Prostate cancer |
| | Tretinoin | acute promyelocytic leukemia |
| | Topotecan | ovarian cancer and lung cancer |
| | Alitretinoin | Kaposi's sarcoma |
| | Mitotane | adrenocortical carcinoma |
| | Pegaspargase | acute lymphoblastic leukemia |
| | Bexarotene | lung cancer, breast cancer, and Kaposi's |
| | | sarcoma |
| | Arsenic trioxide | acute myeloid leukemia |
| | Gefitinib | advanced or metastatic non-small cell lung |
| | | cancer |
| | Bortezomib | relapsed multiple myeloma and mantle cell |
| | | lymphoma. |
| | Erlotinib | non-small cell lung cancer, pancreatic cancer |
| | | and several other types of cancer. |
| | Anagrelide | Chronic myelogenous leukemia |

The antitumor agents disclosed above generally can be classified into the groups described below.

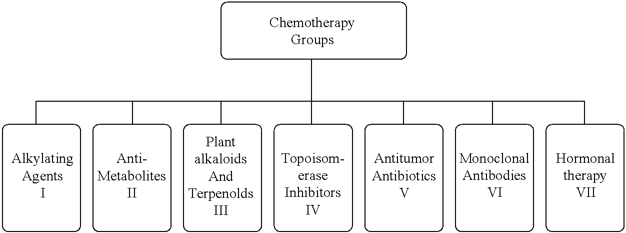

Commercial sources, routes when used at the Maximum Tolerated Dose (MTD) and frequency of administration and dosages for the above-identified antitumor agent can be found in numerous sources such as the Physicians Desk Reference (PDR, 63^{rd} edition, 2008, Thomson Healthcare, Inc., Montvale, N.J.) and The Merck Manual (The Merck Manual, 14^{th} edition, M. J. O'Neil editor, Whitehouse Station, N.J. 20011).

The present invention is directed to methods for increasing the antitumor effects or efficacy of Sindbis virus vectors and pharmaceutical formulation for use in the methods. In one preferred embodiment, Sindbis virus vectors are administered in combination with standard chemotherapeutic agents in high concentrations by bolus administration known as the maximum tolerated dose or MTD or metronically, as defined below. As shown below in Examples 1-4, Sindbis viral vector therapy synergized with the well known antitumor agents CPT-11, Cisplatin and Paclitaxel when administered in this fashion. Given the fact that these agents kill tumor cells by different mechanisms of action (Cisplatin is an alkylating agent, Paclitaxel stabilizes microtubules and CPT-11 is a topoisomerase I inhibitor) it is believed that Sindbis virus vectors will synergize with a wide variety of agents.

In another preferred embodiment, treatment of tumors with Sindbis viral vectors which carry a gene encoding the angiogenic factor VEGF are used to treat tumors. Surprisingly these vectors synergized with chemotherapeutic agents to kill tumor cells. This discovery was based on the observation that tumor blood vessels are less organized and unusually leaky compared to normal blood vessels and that the vascular endothelial growth factor VEGF increased vascular leakiness. The present inventors discovered that specific infection of tumor cells by Sindbis virus vectors was directly correlated with vascular leakiness in tumors. Furthermore, by enhancing tumor vessel leakiness using a Sindbis virus vector carrying a VEGF gene, or co-treatment using chemotherapeutic agents such as Paclitaxel or Cisplatin. greatly enhanced vector delivery and killing of tumor cells. This effect was found using replication defective (RD) and replication competent (RC) Sindbis virus vectors. Therefore, this embodiment is directed to treating mammals harboring tumors by administering an amount of (a) a Sindbis virus vector carrying a VEGF gene and (b) an antitumor agent, wherein the amount of (a) and (b) in combination are effective to treat the tumor. The nucleotide sequence of the human VEGF gene is shown in Example 5 below and the mouse VEGF gene, is shown in Example 6 below. Sindbis virus vectors containing the VEGF gene can be constructed as described in Example 5 below. Antitumor agents for use in this embodiment of the present invention in terms of promoting vascular leakiness for enhanced vector delivery are those designed to target rapidly dividing cells. A broad spectrum of such agents includes alkylating agents (Group I), anti-metabolites (Group II), plant alkaloids terpenoids (Group III) and Topoisomerase inhibitors (Group IV). Particularly preferred agents include Paclitaxel, CPT-11 and Cisplatin.

In light of the above, in another preferred embodiment, the present invention is directed to Sindbis virus vectors comprising a VEGF gene. The Sindbis virus vectors can be replication defective (RD), or comprise a chimeric Sindbis E2 envelope protein. The RD vectors are used to treat tumors which express greater amounts of LAMR than normal cells of the same lineage. Sindbis vectors which comprise a chimeric Sindbis E2 envelope protein can be used to treat tumors which express tumor specific cellular targets. These vectors can be formulated into pharmaceutical formulations or dosage forms which contain pharmaceutically acceptable carriers, excepients or diluents.

The effective amount of the Sindbis virus vector for use in this embodiment broadly ranges between about 10⁶ and about 10¹² virus particles per treatment. For replication competent vectors, the effective amount will range between about 10⁶ and about 10⁸ virus particles per treatment. For chimeric envelope and replication defective vectors, the effective amount will range between about 10¹⁰ and about 10¹² vector particles per treatment.

In a particularly preferred embodiment RD Sindbis virus vectors are used first to deliver the VEGF gene to tumor cells which insures high levels of expression of VEGF at initial infection sites. Such short term limited VEGF expression prevents tumor related angiogenesis and enhanced tumor growth which can result from prolonged expression of VEGF. This is then followed by administration of an amount of a RC Sindbis virus vector effective to treat the tumor. The enhanced blood vessel permeability allows for increased replication of the RC Sindbis virus vector in tumor cells and the death thereof. At later times, post initial RC vector infection, an antitumor agent such as Paclitaxel can be administered resulting in further vascular leakiness without increasing angiogenesis and provides for better therapeutic effects of the RC vector. The RC vector can also be administered multiple times.

In addition, the present inventors have discovered that Sindbis virus vector antitumor therapy is particularly effective when combined with metronomically administered chemotherapeutic agents. Conventional chemotherapy involves the administration of high doses of the agents delivered by bolus administration to patients, known as the maximums tolerated dose (MTD) which requires 2-3 week breaks between successive cycles of administration to allow recovery from myelosuppression. Metronomic administration involves administering substantially lower doses of chemotherapeutic agents (less than 50% of the MTD and preferably between about 10% and about 50% of the MTD) on a frequent schedule (weekly, several times a week or daily) as described in Kerbel et al., Nature Review/Cancer vol. 4, p 423-435, 2004. For example, as shown below in Example 4, the MTD of Paclitaxel is usually 175 mg/mm² in humans given once every 2-3 weeks. However, when given at a dose of 16 or 48 mg/mm2 on days 1, 3 and 6 Paclitaxel caused vascular insult and enhanced tumor vascular leakiness. The vascular leakiness caused by the VEGF gene product promoted RC-Sindbis replication and enhanced tumor cell killing. In addition, the MTD for Cisplatin is 100 mg/mm². Metronomic administration of Cisplatin at 4 or 12 mg/mm² on days 1-4 synergized with Sindbis virus vector infection produces enhanced tumor cell killing. Therefore, substantially lower doses of chemotherapeutic drugs can be administered without diminishing their efficacy. Various metronomic treatment regimens are described in Kerbel et al. cited above.

Another embodiment of the present invention combines metronomic chemotherapeutics with Sindbis virus vectors for treating tumors which are resistant to a chemotherapeutic agent. In this embodiment, the tumor cells are resistant to cell killing by an antitumor agent which was administered at the MTD. In this embodiment the same antitumor agent is administered metronimically with Sindbis/VEGF vectors. One immediate advantage is that metronomically administered chemotherapeutics induce damage to tumor blood vessels and increase vascular permeability for vector delivery. Viral vectors retain efficacy in killing tumors that have developed resistance to conventional chemotherapeutic regimens. Cancer cells can easily evade several chemotherapeutic drugs by modulating expression of a single gene. However, since viral vectors are designed to selectively target cancer cells via tumor specific surface proteins (e.g., LAMR) that are important for cancer cell proliferation or survival, it is less likely that tumor cells will develop resistance to viral vectors.

In another embodiment, RD vectors carrying the VEGF gene can be used with metronomic chemotherapy regimens. However, due to the inability of the vectors to replicate, repetitive treatment with RD Sindbis virus vectors may be necessary to achieve therapeutic effects. In this embodiment, alternate day administration of the vector and the chemotherapeutic agent is preferred.

The data presented herein supports the notion and demonstrates that local modulation of vascular leakiness in tumors with a VEGF expressing Sindbis virus vector further enhances its antitumor efficacy. Another benefit of using metronomic agents with Sindbis/VEGF vector is that the anti-angiogenic effect of chemotherapeutic drugs could counteract any residual pro-angiogenic property of the administered VEGF. In a preferred embodiment the metronomic agents and VEGF synergize to enhance vascular permeability for oncolytic RC sindbis vector propagation and dispersal within the tumor tissue. In a particularly preferred embodiment, the RC Sindbis vim vector carries a payload which causes antitumor effects which are not related to vascular leakiness, such as cytokine genes (IL-12 or IL-15), or pro-drugs and genes (such as Ganciclovir and HSV-tk.

In summary, the combined therapy takes advantage of the efficient anti-angiogenic property of chemotherapeutics and specific antitumor capability of Sindbis virus vectors and provides new hope for cancer patients with relapsed disease due to acquired resistance after conventional MTD chemotherapy.

The present invention is set forth below in examples that are intended to further describe the invention without limiting the scope thereof.

### Example 1

Pursuant to the present invention, treatment of tumor-bearing mice with CPT-11 in combination with Sindbis virus vectors significantly prolonged survival in the treated mice. Untreated mice survived for about 4 weeks after implantation of the tumor cell. Mice treated with Sindbis/LacZ vectors survived for an additional 10 days and those treated with CPT-11 alone survived for an additional 15 days. By day 57 all mice treated with either single therapy died. However, mice treated with both CPT-11 and Sindbis virus vectors survived for much longer periods of time. Surprisingly, about 35% of the CPT-11 plus Sindbis virus vector treated mice appeared to be cured of the cancer. In one experiment 35% of the treated mice were tumor free through 206 days post-treatment and in a second experiment, through 127 days at the time of this writing. This is an unprecedented result in that it has never been possible before the present invention to prolong the survival of mice suffering from aggressive tumors such as the ones used herein for such long periods of time.

In the examples below, the following materials and methods were used.

### Cell Lines

ES2 cells were obtained from the American Type Culture Collection (Manassas, Va.) and were cultured in McCoy's 5A medium (Mediatech, Inc., Herndon, Va.) supplemented with 10% fetal bovine serum. ES2/Fluc cells are derived from the ES2 line by stable transfection of a plasmid, pIRES2-Fluc/EGTP, as described previously. (5)

### Sindbis Vectors

Sindbis/Lacz vectors were produced by electroporation of replicon RNA (SinRep5/LacZ) and helper RNA (DH-BB) into BHK cells, as described previously. (5)

### Animal Models

C.B-17 SCID mice (Taconic, Germantown, N.Y.) were bred with a plasma esterase deficient mouse model—Es1^{c} Foxn1^{nu}/J (Jackson laboratory, Bar Harbor, Me.). Es1^{c} mice lack a plasma esterase that can activate CPT-11 into the much more potent SN-38. Since humans lack a similar plasma esterase, Es1^{c} mice were chosen as the mouse model for all the CPT-11 experiments. Offspring (F2 generation) were typed, and mice homozygous for both the SCID and Es1^{c} phenotypes were bred to generate Es1^{c}/SCID mice. Briefly, blood was collected from F2 mice, and was tested by FACS analysis to determine T and B cell levels, and the plasma esterase activity was measured using a nitrophenyl acetate assay (Spectrum, Gardena, Calif.).

Sindbis+CPT-11 ES2/Fluc tumor survival experiment: Female Es1^{c/}SCID mice (6-12 weeks old) received intrapertioneal injections 1.5×10⁶ ES2/Fluc cells on day 0, and tumor growth was validated by imaging the mice on day 4. Briefly, the bioluminescent tumors were imaged using the IVIS® spectrum system (Caliper Life Sciences, Hopkinton, Mass.). Mice received i.p. injections of 0.3 mL of 15 mg/mL D-luciferin (Gold Biotechnology St. Louis, Mo.), and were anesthetized with 0.3 mL of Avertin (1.25% of 2,2,2-tribromoethanl in 5% t-amyl alcohol). The mice were then imaged for 15 seconds (high resolution binning; field of view D).

Tumor-bearing mice were then divided into 4 groups, with 10 mice per group, and the treatment started on day 5. Group 1 received no treatment, group 2 received Sindbis/LacZ treatment only; group 3 received CPT-11 (Irinotecan Hydrochloride Injection) treatment only; and group 4 received Sindbis/LacZ plus CPT-11 treatment. The mice were treated 4 times a week through i.p. injections, for 5 weeks, with the following doses: Sindbis/LacZ: ˜10⁷ plaque-forming units in 0.5 mL of OptiMEM I; CPT-11: 15 mg/kg in 0.25 mL PBS. The survival experiment was repeated twice. In these experiments, the Sindbis vectors were administered first followed by CT-11 four hours later.

Sindbis+Taxol® ES2/Fluc tumor growth and survival experiment: Female SCID mice (8-12 weeks old) received intraperitoneal injections of 4×10⁶ ES2/Fluc cells on day 0, and tumor growth was validated by imaging the mice on day 1. For quantitative analysis of tumor growth, day 1 tumor load signal was set as 100% for each individual mouse for comparison with subsequent images. Mice (nine per group) were treated with Sindbis/LacZ (I.P.) daily from day 1 to day 11, and with Taxol® (0.4 mg/mouse) on day 3, 6 and 10. Mice were imaged on days 1, 3, 6, 10, and 46. A tumor free mouse was used as a negative control.

### Example 1

### The Survival of Tumor-Bearing Mice is Greatly Prolonged by Combining CPT-11 and Sindbis Vectors

Without treatment, Es1^{c}/SCID mice bearing ES2/Fluc tumors survived for approximately 4 weeks. Mice treated with Sindbis/LacZ survived for approximately 10 days longer, and mice treated with CPT-11 survived for an additional ˜15 days. By day 57, all of the mice that were treated with either single therapy had died. The mice that were treated with both CPT-11 and Sindbis survived for longer, and significantly about 35% appear to have been cured of the cancer (although some of them seemed to have a low number of residual luminescent cells—see below). This experiment was repeated twice. Data from the first experiment was collected through 206 days, while data from the second experiment is available only through 127 days at the time of this writing. In both experiments there was a marked benefit over all other groups in the survival of the group treated with both CPT-11 and Sindbis, although tumor progression occurred faster in the second experiment, leading to a lowered incidence of survival. Seven out of 10 mice survived for substantially longer than the control or singly treated mice.

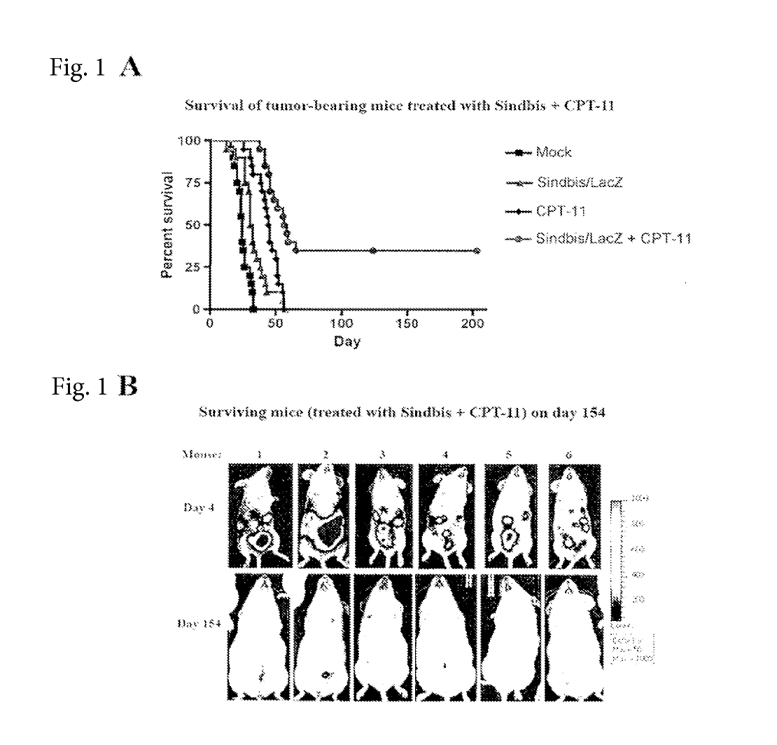

The 7 surviving mice from both experiments appeared to be healthy. The mice from the first experiment were imaged on day 4 and on day 154 after injecting the tumors (FIG. 1B). Of these 6 mice, 3 appeared to be completely tumor-free. The other 3 appeared to have a relatively small number of residual luminescent cells (presumably ES2/Fluc cells) near the injection site. But these luminescent cells (FIG. 1B, bottom) were much smaller than the tumors that were imaged on day 4 (FIG. 1B, top). Furthermore, these cells didn't seem to be growing (data not shown). Most importantly, the mice appeared to be completely healthy, as did the surviving mouse from the second experiment.

### Example 2

### Combining Paclitaxel (Taxol®) and Sindbis Vectors Also Prolongs the Survival of Tumor-Bearing Mice

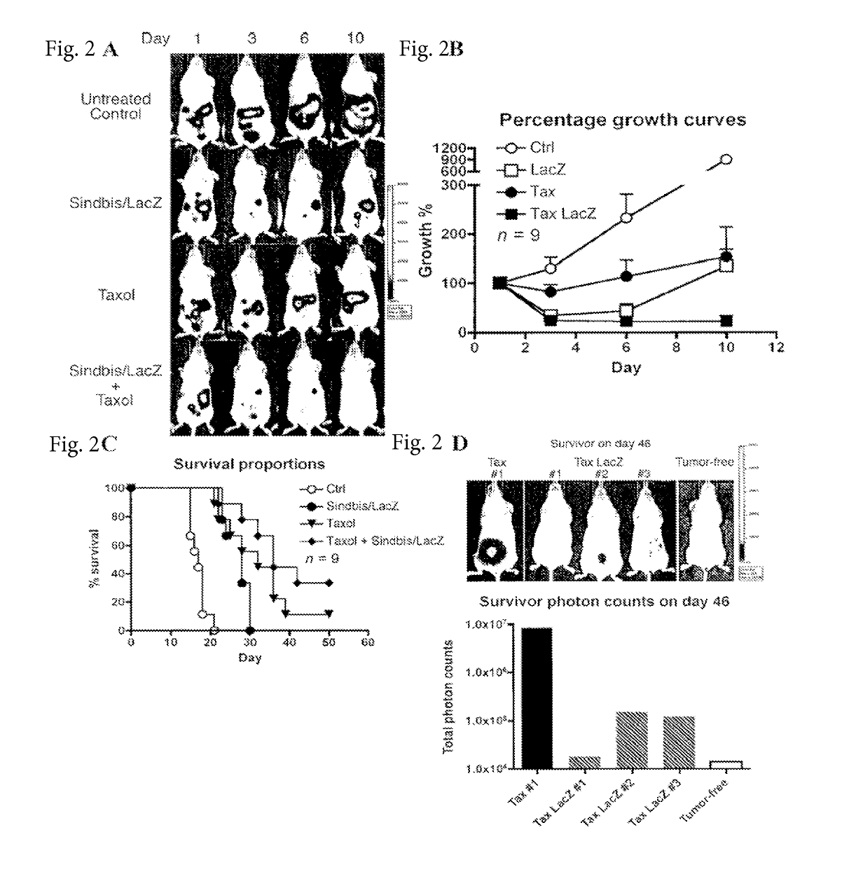

In order to test if the results obtained from treatment with CPT-11+Sindbis also occur with other chemotherapeutic drugs, the effect of Sindbis treatment plus Taxol® on tumor-bearing mice was tested. As with the CPT-11 experiments, the results show that the combination of the two therapies has a stronger therapeutic effect than the single treatments. The tumor load in double-treated mice was lower than in single-treated or control mice when they were imaged on day 3, 6 and 10 (FIG. 2A; quantified in FIG. 2B). In addition, the survival of the double-treated mice was prolonged compared to single-treated and control mice (FIG. 2C). Lastly, the surviving mice were imaged again on day 46, and the double-treated mice were shown to still have a low tumor-load (FIG. 2D).

This set of experiments illustrated that the combination of Taxol® and replication-defective (RD) Sindbis vector achieved very impressive therapeutic results. The combined therapy dramatically reduced tumor burden as indicated in FIG. 2A. It is worthy to note that both Tax and RD-LacZ single treatment groups still show tumor growth, while the group treated with the combined therapy (Tax RD-LacZ) demonstrated complete tumor growth suppression. The treatments were only administered for 11 days. Vector was administered from day 1-11, Tax on day 3, 6 and 10, and the imaging on day 10 indicated very little tumor in the animal. The animals which survived were imaged on Day 46 which strongly suggested synergism between Sindbis vector and chemotherapeutic agent. On day 46 one mouse survived in the Tax group and three in Tax-LacZ group (see FIG. 2B). By then the treatments had been stopped for 35 days which provided sufficient time for any residual tumor in the animal after treatments stopped on day 10, to grow and to be visualized by IVIS® imaging. As shown in FIG. 2C, the Tax survivor had much higher tumor levels than the other three Tax-LacZ survivors. One Tax-LacZ mouse showed undetectable tumor levels in comparison with a tumor-free mouse that served as a negative imaging control.

### Discussion

Presented herein are data which show that a combinatorial anti-cancer approach using chemotherapy and Sindbis vectors is an effective way to treat ES2/Fluc tumor-bearing mice using two different chemotherapeutic agents. Combinatorial therapy can result in antagonistic, additive, or synergistic effects. Synergism occurs when one or both of the treatments enhances the other treatment. Based on the data herein combinatorial therapy using chemotherapy and Sindbis vector treatment enhances the susceptibility of tumor cells to the chemotherapeutic agents and/or to Sindbis vector treatment.

The ES2/Fluc tumor model was chosen because it is a well-established human tumor model, and because it has previously been used by the present inventors ES2/Fluc tumors are partially susceptible to treatment with Sindbis vectors, but the treatment can only prolong the survival of mice by 1-2 weeks. In order to improve this treatment, the chemotherapeutic agent CPT-11 was added to determine if it could enhance the effect of the Sindbis treatment. The results indicated that indeed CPT-11 enhanced the Sindbis treatment. Surprisingly, a significant percent of the mice seemed to be cured of the tumor, a result that was never seen before in this tumor model with any other treatment or combination of treatments. Significantly, none of the single-treatment mice survived for longer than 57 days. indicating that combining the two therapies is needed to achieve a substantial survival prolongation.

Results from a second experiment using the chemotherapeutic agent Taxol® plus Sindbis vectors showed that other antitumor agents could also work effectively in combination with Sindbis virus vector treatment.

### References for Examples 1 and 2

[1] W Scheithauer et al. Randomized Multicenter Phase II Trial of Two Different Schedules of Capecitabine Plus Oxaliplatin as First-Line Treatment in Advanced Colorectal Cancer. Journal of Clinical Oncology, Vol 21, Issue 7 (April), 2003: 1307-1312

[2] D C Bodurka et al. Phase II Trial of Irinotecan in Patients With Metastatic Epithelial Ovarian Cancer or Peritoneal Cancer. Journal of Clinical Oncology, Vol 21, Issue 2 (January), 2003: 291-297

[3] Y Xu et al. Irinotecan: mechanisms of tumor resistance and novel strategies for modulating its activity. Annals of Oncology 13: 1841-1851, 2002

[4] A T Cheung et al. Paclitaxel (Taxol): an inhibitor of angiogenesis in a highly vascularized transgenic breast cancer. Cancer Biother Radiopharm. 1999 February; 14(1): 31-6.

[5] J C Tseng et al. In vivo antitumor activity of Sindbis viral vectors. J Natl Cancer Inst. 2002 Dec. 4; 94(23): 1790-802.

[6] K S Wang et at. High-affinity laminin receptor is a receptor for Sindbis virus in mammalian cells. Journal of Virology, August 1992, p. 4992-5001

[7] J C Tseng J C et al. Using sindbis viral vectors for specific detection and suppression of advanced ovarian cancer in animal models. Cancer Res. 2004

[8] D Kim et al. Efficacy with a Replication-selective Adenovirus Plus Cisplatin-based Chemotherapy: Dependence on Sequencing but not p53 Functional Status or Route of Administration. Clinical Cancer Research Vol. 6, 4908-4914, December 2000

[9] D Hoffmann et al. Synergy between expression of fusogenic membrane proteins, chemotherapy and facultative virotherapy in colorectal cancer. Gene Therapy 13, 1534-1544. doi:10.1038/sj.gt.3302806; published online 22 Jun. 2006

[10] M D Steller et al. Inhibin Resistance Is Associated with Aggressive Tumorigenicity of Ovarian Cancer Cells. Molecular Cancer Research 3: 50-61 2005

### Example 3

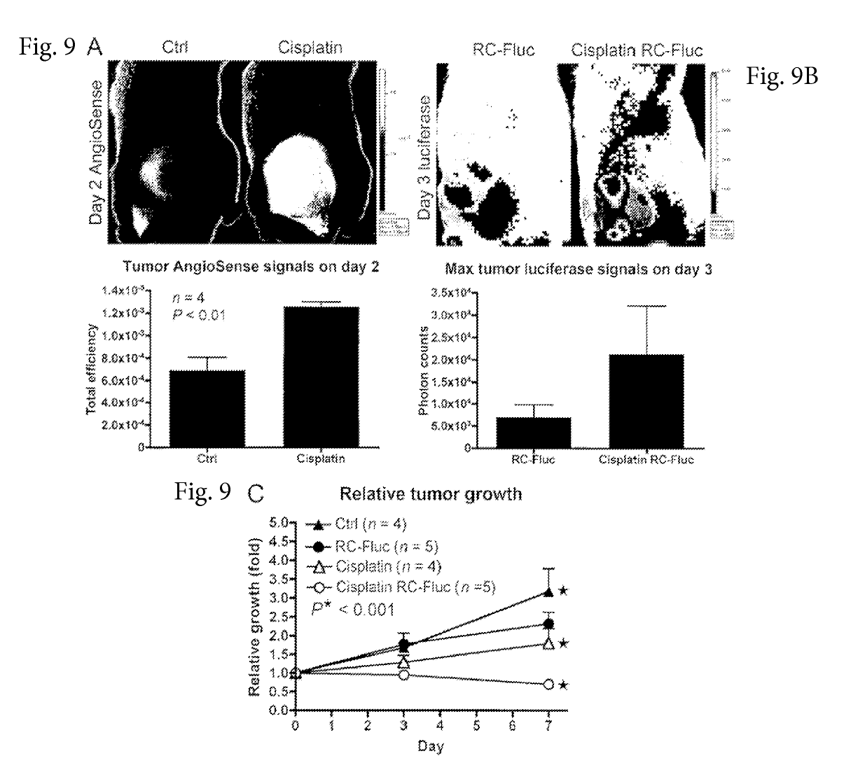

Female Es1/SCID mice were inoculated intraperitoneally with 5 million luciferase-expressing Mia Paca cells (a model for pancreatic cancer) on day 0. Mice were then divided into 4 groups: Mock (untreated), Sindbis/LacZ treated, CPT-11 treated, and Sindbis/LacZ+CPT-11 treated. The mice were treated 4 times a week, for 2 weeks, and then the treatment was stopped. The 3 double-treated mice appear to be tumor-free in all of the images taken since day 18. All of the untreated and single-treated mice have tumors that appear to be growing (FIG. 9).

### Example 4

### Introduction

The goal of cancer gene therapy is to achieve specific and efficient delivery of gene therapy vectors to tumor cells while reducing the impact of unwanted toxicity, associated with the vector of choice, to normal tissues. In addition, to maximize therapeutic effects, an ideal vector system should be able to achieve systemic delivery, via the bloodstream, to distal or metastasized tumor cells. Several viral vector systems have been developed to specifically transduce tumor cells by modification of viral structural proteins (1-4), or to selectively replicate in tumors by taking advantage of tumor specific signaling pathways (5, 6). Currently, however, only a few viral vector systems, among which is Sindbis vector (7), are capable of systemic delivery without dramatically reducing efficacy. Along with tumor specificity and systemic delivery, a vector must efficiently penetrate tumor vascular structures in order to reach and transduce cancer cells.

Tumor growth depends upon angiogenesis and many cancer therapy agents have been developed to target newly formed tumor blood vessels (8). Unlike normal vessels, the endothelium cells in tumor vessels are less organized and unusually leaky (9). Abnormal blood vessel leakiness has been known in tumors, and higher levels of leakiness correlate with histological grade and malignancy (10). The vessel leakiness can cause extravasations of plasma proteins and even erythrocytes in some extreme cases (hemorrhage). These phenomena have been supported by evidence from several experimental tumors, including extravasations of small soluble tracers such as radioisotopes, albumin, dextran, as well as larger particles such as colloidal carbon and liposomes up to 2 μm in size (11-13). Intratumoral hemorrhage is an extensive form of vascular leakiness, which ranges from scattered extravasated erythrocytes to a blood lake, consisting of larger collections of erythrocytes surrounded by tumor cells (14, 15). Such vessel leakiness may be the direct result of hyperactive angiogenesis and vascular remodeling in tumors. On the other hand, increased leakiness of tumor vessels allows deeper penetration and may provide a means to selectively deliver cancer therapeutic agents into tumor tissues. In particular, tumor vessel leakiness should play an important role in the delivery of larger therapeutic agents, such as oncolytic viruses, into tumors.

Our previous findings indicate that vectors based on the Sindbis virus are capable of systemic tumor targeting via the bloodstream (7). The specific targeting is attributed to higher expression levels of high-affinity laminin receptor (LAMR) on cancer cells, which promotes cell adhesion, invasion and metastasis (16). After tumor transduction, the replication-defective (RD) vector system is capable of efficient transgene expression using a viral specific subgenomic promoter. With suitable reporter genes, we have demonstrated the use of RD Sindbis viral vector to detect and monitor tumors in small laboratory animals using molecular imaging methods, such as bioluminescence (7) and positron emission tomography (PET) (17). Also, the fact that Sindbis transduction causes tumor death by inducing apoptosis, even without adding payload genes, makes Sindbis derived vectors promising therapeutic agents for cancer therapy (18, 19).

Little is known about the correlation between vector delivery/transduction kinetics and tumor vascular leakiness. In this report, we provide in vivo bio-optical imaging evidence that specific transduction of Sindbis vector directly correlates with vascular leakiness in tumors. Furthermore, enhancing tumor vessel leakiness using a vector carrying a vascular endothelium growth factor gene (VEGF) or co-treatment using chemotherapy agents, such as Paclitaxel and Cisplatin, greatly enhances vector delivery and transduction in tumors. Our results suggest that, in addition to strategies currently used involving tumor specific surface markers or cancer-type specific signaling features, modulation of tumor vascular leakiness could provide an additional layer of tumor specificity. Thus, the capability to manipulate tumor vessel leakiness could be an important tool to achieve improved cancer gene therapy using oncolytic viruses, especially due to their intrinsically larger size compared with other smaller agents.

### Materials and Methods

### Cells and Vector Preparation

Hamster BHK and mouse N2a cells (American Type Culture Collection, Manassas, Va.) were maintained in αMEM (JRH Bioscience, Lenexa, Kans.) with 5% FBS and in Eagle-modified media (MEM, JRH Bioscience) with 10% FBS, respectively. ES-2/Fluc cells were derived from human ES-2 ovarian cancer cells (20), and were maintained in McCoy's 5A medium (Mediatech, Inc., Herndon, Va.) with 10% FBS.

Constructions of RD-Sindbis/Fluc and /LacZ are previously described (7). RD-Sindbis/mPlum was constructed by insertion of a DNA fragment encoding the mPlum protein (from pmPlum plasmid, Clontech Laboratories Inc., Mountain View, Calif.) into the pSinRep5 replicon plasmid at the PmlI site. We performed similar procedures to generate RD-Sindbis/VEGF using a DNA fragment from pBLAST49-mVEGF plasmid (InvivoGen Inc., San Diego, Calif.). Production of Sindbis vector particles was achieved by in vitro transcription of replicon (from pSinRep5) and helper (from pDH-BB) RNAs, followed by electroporation of both replicon and helper RNAs into BHK cells as previous described (16). A replication-competent (RC) Sindbis/Fluc vector was constructed by insertion of a second subgenomic promoter and viral structural genes downstream of firefly luciferase gene as previously described (21).

### Imaging

Qtracker® 800 quantum dot was obtained from Molecular Probes Inc. (Eugene, Oreg.). AngioSense® 750 was purchased from VisEn Medical (Bedford, Mass.). The fluorescent imaging was done using IVIS® Spectrum imaging system (Caliper Life Sciences, Inc., Hopkinton, Mass.). Each image of indicated excitation/emission matrix was acquired for 1 sec at aperture setting of f4. The raw sequential imaging data were analyzed using the Living Image® 3.0 software (Caliper Life Science, Inc.) to unmix concentration maps for Qtracker® and AngioSense.

All animal experiments were performed in accordance with NIH and institutional guidelines. BHK cells (1.5×10⁶/mouse) were s.c. inoculated into SCID mice (female, 6-8 week old, Taconic, Germantown, N.Y.). The mouse neuroblastoma tumors were induced by s.c. injection of 1.5×10⁶ N2a cells into SCID mice 13 days prior to treatments. For better visualization, we remove excessive fur on the skin over the tumor and its surrounding region. The setting for dual mPlum/AngioSense imaging is as following: ex605/em660, 680 and 700 nm, followed by ex745/em800, 820 and 840 nm. Bioluminescent imaging of luciferase activities was performed as described before (7). Tumor sizes were measured using the formula: π/6×length (mm)×width (mm)².

### Statistical Analysis

We used Prism® 4 for Macintosh (GraphPad Software, Inc., La Jolla, Calif.) to perform statistical analysis of our data. Quantitative imaging data and tumor growth curves were analyzed using Two-way ANOVA. All P values generated were in two-tailed.

### Results

### Near-Infrared (NIR) Fluorescent Imaging of Tumor Vessel Leakiness In Vivo

In order to visualize tumor vessels and vascular leakiness, we used two different near-infrared (NIR) fluorescent probes, Qtracker® and AngioSense®, for in vivo molecular imaging of tumor vasculature. Qtracker® is a non-targeted fluorescent nanoparticle (20-50 nm in diameter) with a broad excitation wavelength (400-700 nm) and an emission wavelength at around 800 nm. The rigid sphere shape of the nanoparticle makes Qtracker® stable in circulation. In addition, the surfaces of these quantum dots are chemically modified to reduce non-specific binding and immune responses, making Qtracker® a useful imaging tool for in vivo imaging of tumor vessels with minimal leakage from the vasculature. In contrast, AngioSense® is a smaller and flexible NIR fluorescent macromolecule (250 k MW). Unlike Qtracker®, AngioSense® has a narrower excitation wavelength at 750 nm and an emission wavelength at around 800 nm. AngioSense® is designed as a NIR imaging probe for vascularity, perfusion and vascular permeability. Although both NIR probes have similar emission wavelength at ˜800 nm, it is possible to distinguish their specific distribution by using different excitation wavelengths (˜500 nm for Qtracker® and ˜750 nm for AngioSense®)).

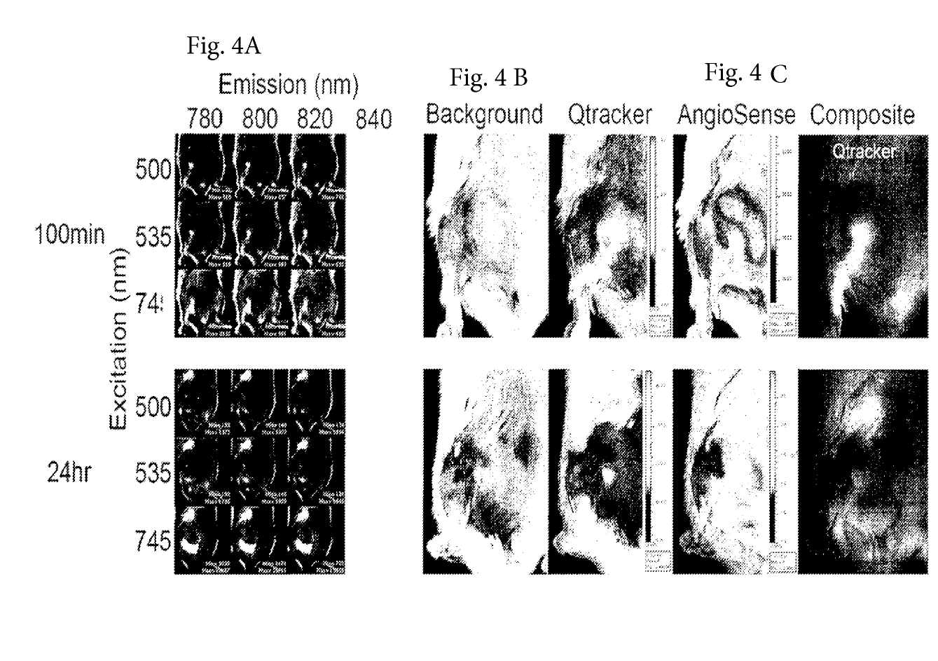

Taking advantage of our IVIS® spectrum imaging system, which is capable or acquiring sequential fluorescent excitation-emission images of the same subject, we intravenously injected the Qtracker®/AngioSense® mixture into a tumor-bearing mouse to determine if we could visualize general tumor vessel structure and vascular leakiness (FIG. 4). A severe combined immunodeficiency (SCID) mouse, bearing a subcutaneous (s.c.) tumor, was used for its known vascular leakiness for Sindbis vector delivery. To visualize general vascular structure, we performed the first sequential imaging matrix 100 min after tracer administration via the tail vein (FIG. 4A). For leakiness imaging, a second imaging matrix was performed 24 hour after tracer injection (FIG. 4A). The reconstructed concentration maps at 100 min indicate that both Qtracker® and AngioSense® signals have similar distribution patterns that identify general vessels in the tumor (FIG. 4B). However, the 24 hr concentration maps suggest that the Qtracker® signals still retain a similar distribution pattern as before, while the AngioSense® develops a more disperse and widespread pattern than the 100 min images, indicating vascular leakiness in these regions (FIG. 40B). In addition, the IVIS® spectrum system is capable of analyzing the excitation-emission matrix and generates a reconstructed concentration map of Qtracker® and AngioSense® in each mouse (FIG. 4C). These data indicate that while AngioSense® is capable of imaging general vascular structure within a short period time (<3 hrs) after its administration, prolonged incubation (≧24 hrs) provides a means to visualize leaky tumor vasculature.

### Sindbis Viral Vector Transduction Correlates With Tumor Vessel Leakiness

Having established the ability to visualize leaky vascular regions in tumors, we tested if there is a correlation between tumor leakiness and Sindbis vector transduction. In the first set of experiments we used a replication-defective vector carrying the mPlum gene. Originally derived from DsRed protein, mPlum fluorescent protein has a red-shifted functional spectrum (ex: 590 nm; em: 650 nm) suitable for in vivo imaging.

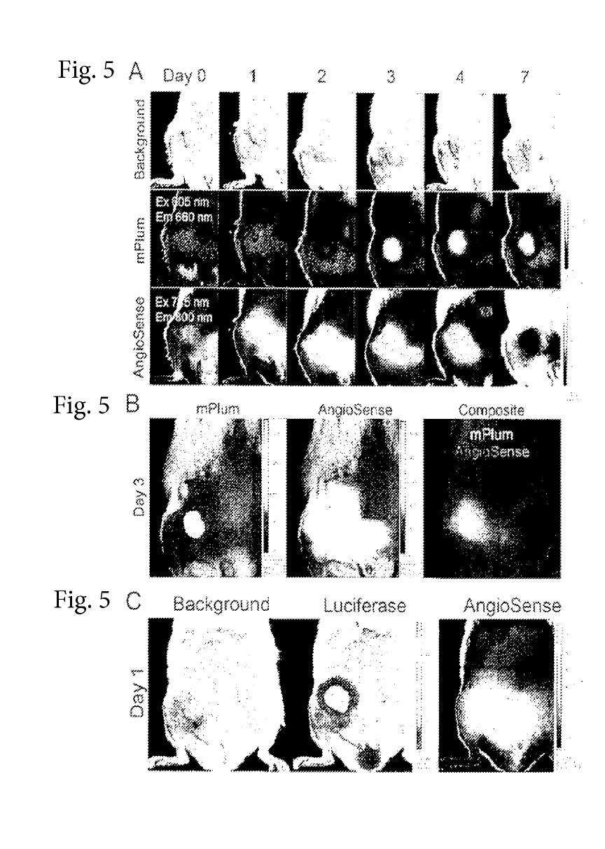

On day 0, a single dose of intravenous (i.v.) RD-Sindbis/mPlum treatment was injected into a SCID mouse bearing a s.c. BHK tumor. The AngioSense® was also i.v. administrated on the same day and the first IVIS® imaging matrix for both mPlum and AngioSense® signals was acquired 2 hrs after AngioSense® injection. Follow-up images were acquired on day 1, 2, 3, 4, and 7. For simplicity FIG. 5A only shows the individual images of the optimal excitation-emission pair for mPlum (ex605/em660 nm) and AngioSense® (ex745/em800 nm). The AngioSense® signal on day 0 only shows the major tumor vessels since the majority of the tracer is still in free circulation. Starting on day 1, as circulating AngioSense® starts to extravasate from leaky blood vessels and is retained in surrounding tumor tissues, we were able to distinguish tumor regions that showed higher vascular permeability.

That none or very little of mPlum signal was detected in the tumor 2-24 hrs was not surprising, since the vector needs some time to amplify sufficient mPlum protein for IVIS® detection. However, on day 2, tumor-specific mPlum signals were observed in tumor regions whose size and shape are very similar to day 1 AngioSense® signals, suggesting that the initial RD-Sindbis/mPlum transduction occurred at the original tumor regions that show high vascular leakiness. On day 2, the AngioSense® signal pattern indicated that the tumor was expanding and there was a region showing exclusion of the probe, which suggested the presence of necrotic tumor tissue with reduced permeability. On day 3 the mPlum signal became very strong and seemed to correlate with the necrotic tumor region. Due to probe excretion from the urinary track (as evident by strong bladder signal on day 2), the AngioSense® signals started to fade away on day 3 and very little remained by day 7. On the other hand, mPlum signals remained in necrotic tumor tissue and were detectable until day 7, suggesting that sufficient mPlum protein, which was produced inside tumor cells after Sindbis/mPlum transduction, remained within the necrotic tissue thereafter.

To verify that the necrotic tumor region was caused by Sindbis/mPlum transduction, we reconstructed the concentration maps of mPlum and AngioSense® using the imaging data set obtained on day 3 (FIG. 5B). As shown in the composite image, the fact that both mPlum and AngioSense® signals are distinctively present strongly suggests that the necrotic region is directly caused by Sindbis transduction.

Unlike the RD-Sindbis/mPlum vector that requires more than 1 day to visualize tumor-specific transduction, a parallel experiment using the RD-Sindbis/Fluc vector indicated that firefly luciferase provided better sensitivity. We were able to detect tumor-specific luciferase signal on day 1. In addition, the luciferase signals correlated nicely with the leaky vasculature as indicated by AngioSense® signals. These results support our hypothesis that vascular leakiness is important for Sindbis vector tumor targeting.

### VEGF Enhances Tumor Vascular Leakiness and Promotes Sindbis Vector Transduction

We tested whether enhancing tumor vascular leakiness would benefit Sindbis vector delivery and transduction in tumors. A replication-defective vector (RD-Sindbis/VEGF) was constructed to deliver a mouse vascular endothelial growth factor (VEGF) gene. Besides playing a key role in regulating blood vessel growth in both normal and pathological conditions, VEGF was first identified as a vascular permeability factor (VPF). VEGF treatments on endothelial cells enable passage of particles of different sizes through vessels by a variety of physical mechanisms. In experimental tumors, the functional limits and defined pore cutoff sizes of transvascular transport induced by VEGF is believed to range from 200 nm to 1.2 μm. This level of vascular permeability would allow larger particles, such as Sindbis viral vectors (˜70 nm in diameter), to extravasate into tumor tissues.

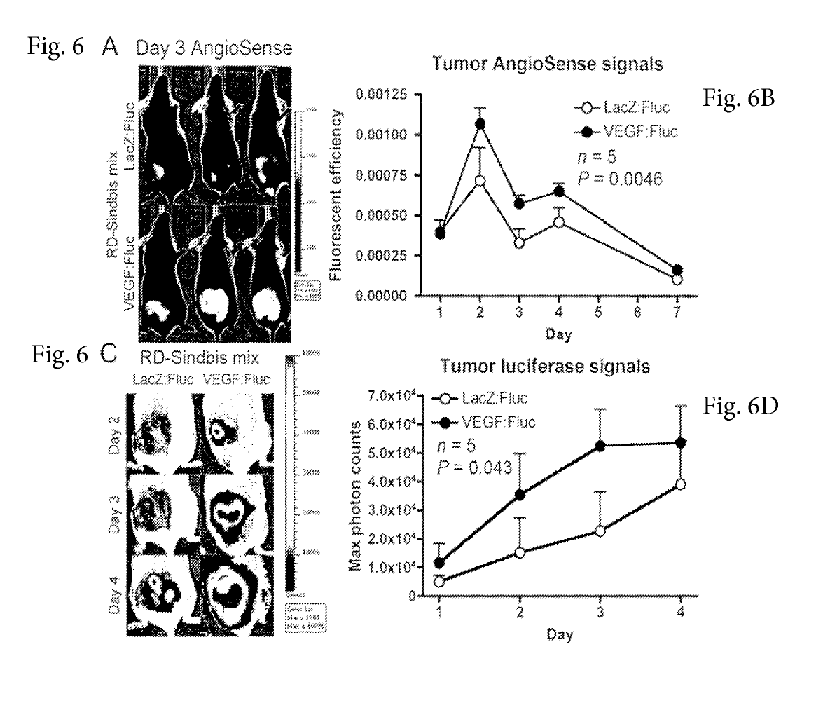

Since the RD-Sindbis/VEGF does not carry a reporter gene for imaging, we used a mixture of RD-Sindbis/VEGF and RD-Sindbis/Fluc vectors (1:1) to evaluate specific tumor transduction in the SCID/BHK s.c. tumor model. A vector mixture of RD-Sindbis/LacZ:RD-Sindbis/Fluc (1:1) was used as a control. intraperitoneal (i.p.) treatments of the RD VEGF/Fluc mixture significantly enhanced tumor vascular leakiness as evidenced by increased AngioSense® signals (FIGS. 6A and 6B). As expected, higher vessel leakiness in RD VEGF/Fluc treated tumors resulted in higher RD-Sindbis/Fluc transduction (FIGS. 6C and 6D). This result supports the idea of modulating tumor vessel leakiness in order to improve viral vector delivery and transduction.

### Chemotherapeutic Agents and VEGF Increase Vessel Leakiness and Enhance Therapeutic Efficacy of Sindbis Vectors

Several chemotherapy agents have been developed for first-line treatments of cancer, including taxanes (Paclitaxel and docetaxel) and platinum-based drugs (Cisplatin, carboplatin, and oxaliplatin). These drugs do not specifically target tumor cells, but rather interfere with cell division. For example, Paclitaxel blocks microtubule dissembly during mitosis. Cisplatin causes DNA damage resulting in cell-cycle checkpoint and apoptosis. Therefore, in addition to cancer cells, these drugs also damage normal dividing cells of tissues with rapid regeneration, such as bone marrow, hair follicles and gut mucosa. As a result, most chemotherapeutic agents have narrow therapeutic indexes due to high host toxicity.

Cancer cells are not the only rapid-dividing cells in tumors. Dividing endothelial cells in growing blood vessels in tumors should also be susceptible to chemotherapeutic agents. Furthermore, as endothelial cells originate from normal host tissues, they are assumed to be more genetically stable and with less genetic defects usually present in cancer cells. This feature makes endothelial cells less likely than cancer cells to develop drug resistance especially after prolonged treatments of chemotherapy. Therefore, cancer cells that are resistant to a particular chemotherapy agent could indirectly respond to the agent through an attack of the tumor vasculature. Damaged tumor blood vessels may result in increased vascular permeability.

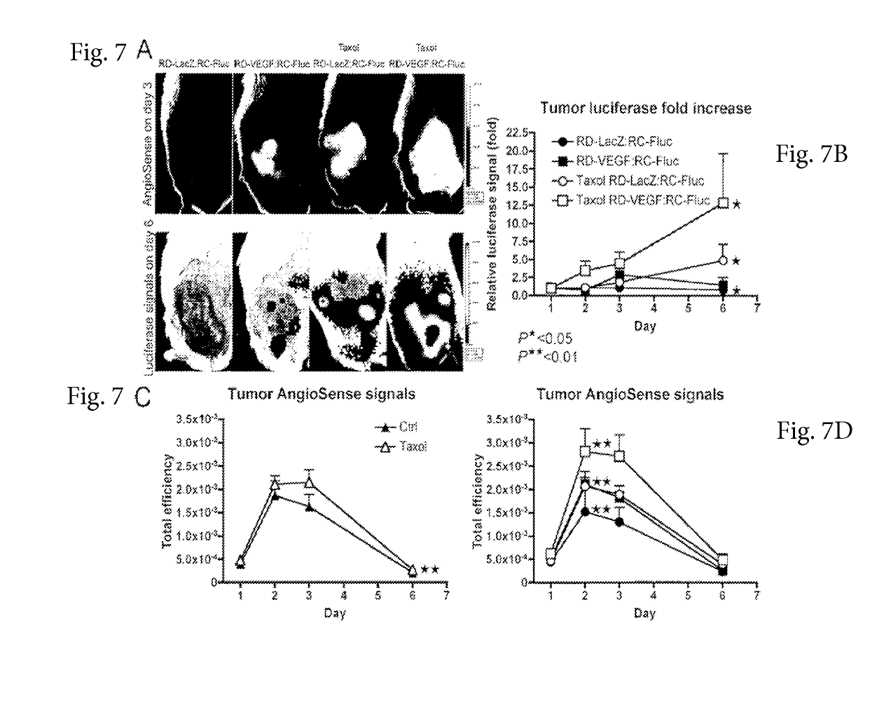

In order to test whether chemotherapeutic agents synergize with Sindbis vector in tumor eradication by modulating vascular leakiness, we used a s.c. mouse N2a neuroblastoma model. Sindbis vector has a lower infectivity in N2a cells compared with BHK cells (about 1000 time less). However, N2a neuroblastoma tumors are well vascularized (FIG. 7A) and therefore are suitable to test any modulation of vascular leakiness that would enhance Sindbis vector transduction. The choice of chemotherapeutic agent is Paclitaxel since it has been shown to inhibit tumor angiogenesis at low concentration and endothelium cells are 10-100 times more sensitive than tumor cells.

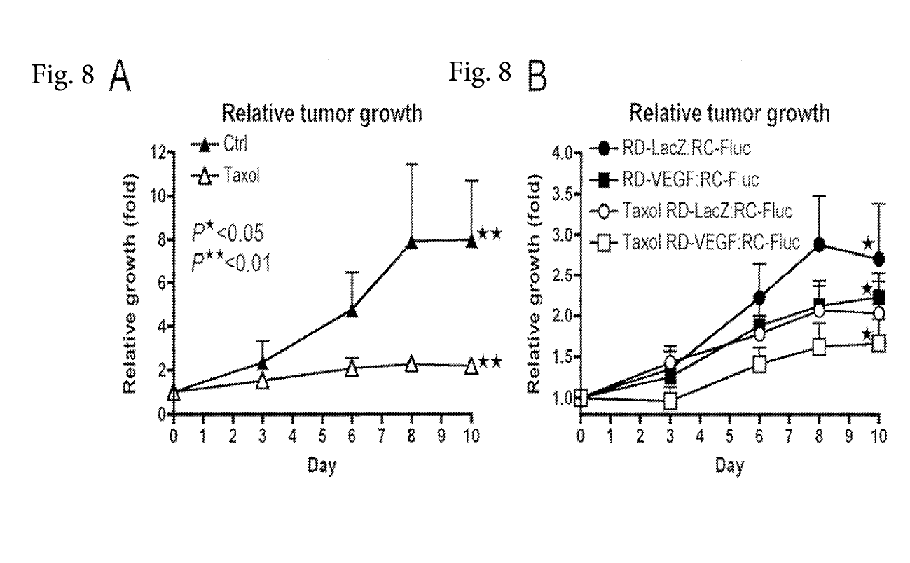

We used a replication-competent (RC) Sindbis/Fluc vector (21) instead of an RD one to further enhance the tumor transduction signal output (FIG. 7B) in N2a tumors. RC vector carries a full set of viral structural genes to support its replication. Specific tumor infection of RC vector could result in oncolytic effects by intratumoral vector replication and amplification. We also tested if the combination VEGF and Paclitaxel further promote RC-Sindbis vector replication in tumors. To ensure temporary expression of VEGF, we used replication defective RD-Sindbis/VEGF mixed with RC-Sindbis/Fluc (1:1). As in BHK tumors, Paclitaxel treatments significantly increased vascular permeability in N2a tumors (FIG. 7C). Together, the drug and VEGF further enhanced vessel leakiness in N2a tumors (FIG. 7D) resulting in improved Sindbis vector transduction (FIG. 7B). Although Paclitaxel treatment alone suppresses tumor growth, the combination treatments improved therapeutic effects of Sindbis vectors (FIG. 8).

We later tested if another chemotherapy agent, Cisplatin, has similar effects if metronomically administrated. Cisplatin treatments significantly increased vascular permeability in s.c. N2a tumors (FIG. 9A). In addition, Cisplatin enhanced the delivery of RC-Sindbis/Fluc and transduction of N2a tumors (FIG. 9B), contributing to better therapeutic efficacy (FIG. 9C). These results support the idea that chemotherapeutic agents may improve the therapeutic outcome of oncolytic viral vectors by enhancing tumor vessel permeability and vector delivery.

### Discussion

In this report, we use novel molecular imaging techniques to visualize the correlation between tumor vascular leakiness and oncolytic vector delivery. Our results indicate that blood vessel permeability in tumors plays a significant role in successful vector targeting. Sindbis virus is considered a small virus with an average size of 60-70 nm in diameter, compared with other viruses recently developed for gene therapy purposes (adenovirus 90-100 nm, vesicular stomatitis virus 65-185 nm, and lentivirus 95-175 nm). Combined with its natural blood-borne capability, the smaller size makes Sindbis vectors suitable for systemic delivery. However, viral vectors are very large in comparison with chemotherapeutic agents. Methods to enhance vessel permeability may dramatically enhance the therapeutic efficacy of viral vectors against cancer. Using bio-optical NIR probes, we can specifically determine vascular leakiness and establish the kinetics of Sindbis vector transduction in tumors. This method should be of significant value for studying physiological conditions in tumors during or after oncolytic viral treatments.

VEGF was first identified as a vascular permeability factor (VPF) (22), and subsequent studies revealed the importance of VEGF in tumor vascular development and angiogenesis. However, the fact that VEGF-induced angiogenesis does not require VEGF-induced vascular permeability suggests that these two functions of VEGF are separate entities (23). In short-term, VEGF-mediated vascular permeability leads to accumulation of a fibrin barrier around tumors (24), which may limit their malignant properties. However, the benefits of prolonged expression of VEGF, by promoting vascular development in tumors, may outweigh the negative impacts of the VEGF-induced vascular leakiness. VEGF modulates endothelial cell-cell junctions, including adherens, tight and gap junctions, via signaling of Src family kinases and/or various protein tyrosine phosphatases (PTP) (25). Here we demonstrate that tumor-specific expression of VEGF improves delivery and replication of oncolytic RC Sindbis viral vectors in tumors. In particular, we used a RD Sindbis vector to deliver VEGF to tumor cells, which ensures temporary expression of VEGF at initial infection sites. Such limited expression could prevent tumor related angiogenesis, as a result of prolonged exposure of VEGF, while providing sufficient vessel permeability to maintain active oncolytic replication of RC Sindbis vectors within tumors (FIG. 5).

By targeting rapidly dividing cells, conventional cytotoxic chemotherapy agents affect not only proliferating tumor cells but also various types of normal cells, such as those of the bone marrow, the hair follicles, the gut mucosa and, more importantly, the endothelium of the growing tumor vasculature. The anti-angiogenic effects of chemotherapy could indirectly contribute to their antitumor efficacy. By administrating such drugs in small doses on a frequent schedule or “metronomically” (weekly, several times a week or daily), their anti-angiogenic effects seem to be enhanced and maintained for prolonged periods (26).

Traditionally, conventional chemotherapy has been administrated at more toxic “maximum tolerated dose” (MTD), which require 2˜3-week breaks between successive cycles of therapy for patients to recover from myelosuppression. However, such long periods of time may cause repair of tumor vasculature, since the proportion of dividing endothelial cells in tumor blood vessels might be too low for the MTD chemotherapy regimen to have significant impact (27). After cancer cells, due to their intrinsic genetic instability, acquire resistance to chemotherapy agents, MTD regimens could counteract the potential benefit of anti-angiogenic effects. By contrast, many studies of preclinical models indicate that metronomic chemotherapy is effective in treating tumors in which the cancer cells have developed resistance to the same chemotherapeutics (28). Thus, metronomic chemotherapy regimens have the advantage of being less acutely toxic, making prolonged treatments and suppressing angiogenesis possible. For example, it has been shown that some metronomic regimens suppress circulating endothelial progenitor cells (27).

Combining metronomic chemotherapeutics with oncolytic vectors might be a promising strategy for cancer treatments. One immediate advantage is that chemotherapeutics induce damages in tumor blood vessels and increase vascular permeability for oncolytic vector delivery. Oncolytic viral vectors should retain efficacy in killing tumors that have developed resistance to conventional chemotherapeutic regimens. Since they are designed to selectively target cancer cells via tumor specific promoters or surface proteins that are important for cancer cell proliferation or survival, it is less likely that tumor cells will develop resistance to viral vectors. On the other hand, it is comparatively easier to acquire resistance to chemotherapeutics. One such example is the up-regulation of multidrug resistant 1 (MDR1) gene in human breast cancers, which encodes the P-glycoprotein drug-efflux pump (29). As a result, cancer cells can easily evade several chemotherapy drugs by modulating expression of a single gene. Therefore, combined metronomic-oncolytic vector regimens may provide new hope for cancer patients with relapsed disease due to acquired resistance after conventional MTD chemotherapy.

Of course, it is possible to envision a mechanism by which chemotherapeutics directly enhance the infectivity of the viral vector to tumor cells instead of targeting tumor vasculature. However, this does not appear to be the case for Paclitaxel, since pretreatment of cancer cells with the drug does not enhance their susceptibility to Sindbis vector (data not shown).

In this report we used an oncolytic vector system based on Sindbis virus to achieve selective targeting and replication in tumors. This vector targets laminin receptor (LAMR) on cancer cells for specific binding and infection (30). Intracellular LAMR precursor (37-Kda LRP) is crucial for cellular ribosomal function (31), while its mature 67-Kda form is important to mediate cancer cell migration and metastasis (32). Furthermore, LAMR seems to be essential for cell survival. The importance of LAMR for oncogenesis makes Sindbis vector suitable for oncolytic purposes.

Our present data supports the notion and demonstrates that local modulation of vascular leakiness in tumors with a VEGF expressing RD vector further enhances its antitumor efficacy. Another benefit of using metronomic agents with Sindbis/VEGF vector is that the anti-angiogenic effect of chemotherapy drugs could counteract any residual pro-angiogenic property of the administrated VEGF. Simultaneously, the metronomic agents and the VEGF synergize to enhance vascular permeability for oncolytic RC Sindbis vector propagation and dispersal within the tumor tissue. In summary, the combined therapy takes advantage of the efficient anti-angiogenic property of chemotherapeutics and specific antitumor capability of oncolytic viral vectors.

### References for Example 4

1. Iijima Y, Ohno K, Ikeda H, Sawai K, Levin B, Meruelo D. Cell-specific targeting of a thymidine kinase/ganciclovir gene therapy system using a recombinant Sindbis virus vector. Int J Cancer 1999; 80(1): 110-8.

2. Ohno K, Meruelo D. Retrovirus vectors displaying the IgG-binding domain of protein A. Biochem Mol Med 1997; 62(1): 123-7.

3. Ohno K, Sawai K, Iijima Y, Levin B, Meruelo D. Cell-specific targeting of Sindbis virus vectors displaying IgG-binding domains of protein A. Nat Biotechnol 1997; 15(8): 763-7

4. Sawai K, Meruelo D. Cell-specific transfection of choriocarcinoma cells by using Sindbis virus hCG expressing chimeric vector. Biochem Biophys Res Commun 1998; 248(2): 315-23.

5. Marcato P, Shmulevitz M, Lee P W. Connecting reovirus oncolysis and Ras signaling. Cell Cycle 2005; 4(4): 556-9.

6. O'Shea C C. Viruses—seeking and destroying the tumor program. Oncogene 2005; 24(52): 7640-55.

7. Tseng J C, Levin B, Ilurtado A, et al. Systemic tumor targeting and killing by Sindbis viral vectors. Nat Biotechnol 2004; 22(1): 70-7.

8. Carmeliet P. Angiogenesis in life, disease and medicine. Nature 2005; 438(7070): 932-6.

9. Ribatti D, Nico B, Crivellato E, Vacca A. The structure of the vascular network of tumors. Cancer Lett 2007; 248(1): 18-23.

10. McDonald D M, Baluk P. Significance of blood vessel leakiness in cancer. Cancer Res 2002; 62(18): 5381-5.

11. Hobbs S K, Monsky W L, Yuan F, et al. Regulation of transport pathways in tumor vessels: role of tumor type and microenvironment. Proc Natl Acad Sci U S A 1998; 95(8): 4607-12.

12. Leunig M, Yuan F, Menger M D, et al. Angiogenesis, microvascular architecture, microhemodynamics, and interstitial fluid pressure during early growth of human adenocarcinoma LS174T in SCID mice. Cancer Res 1992; 52(23): 6553-60.

13. Yuan F, Dellian M, Fukumura D, et al. Vascular permeability in a human tumor xenograft: molecular size dependence and cutoff size. Cancer Res 1995; 55(17): 3752-6.

14. Liwnicz B H, Wu S Z, Tew J M, Jr. The relationship between the capillary structure and hemorrhage in gliomas. J Neurosurg 1987; 66(4): 536-41.

15. Van den Brenk H A, Crowe M, Kelly H, Stone M. G. The significance of free blood in liquid and solid tumours. Br J Exp Pathol 1977; 58(2): 147-59.

16. Tseng J C, Levin B, Hirano T, Yee H, Pampeno C, Meruelo D. In vivo antitumor activity of Sindbis viral vectors. J Natl Cancer Inst 2002; 94(23): 1790-802.

17. Tseng J C, Zanzonico P B, Levin B, Finn R, Larson S M, Meruelo D. Tumor-specific in vivo transfection with HSV-1 thymidine kinase gene using a Sindbis viral vector as a basis for prodrug ganciclovir activation and PET. J Nucl Med 2006; 47(7): 1136-43.

18. Venticinque L, Meruelo D. Sindbis viral vector induced apoptosis requires translational inhibition and signaling through Mcl-1 and Bak. Submitted 2008.

19. Levine B, Huang Q, Isaacs J T, Reed J C, Griffin D E, Hardwick J M. Conversion of lytic to persistent alphavirus infection by the bcl-2 cellular oncogene. Nature 1993; 361(6414): 739-42.

20. Tseng J C, Hurtado A, Yee H, et al. Using sindbis viral vectors for specific detection and suppression of advanced ovarian cancer in animal models. Cancer Res. 2004; 64(18): 6684-92.

21. Tseng J C, Daniels G, Meruelo D. Controlled propagation of replication-competent Sindbis viral vector using suicide gene strategy. Gene Ther 2008.

22. Senger D R, Galli S J, Dvorak A M. Perruzzi C A, Harvey V S, Dvorak H. F. Tumor cells secrete a vascular permeability factor that promotes accumulation of ascites fluid. Science 1983; 219(4587): 983-5.

23. Eliceiri B P, Paul R, Schwartzberg P L, Hood J D, Leng J, Cheresh D A. Selective requirement for Src kinases during VEGF-induced angiogenesis and vascular permeability. Mol Cell 1999; 4(6): 915-24.

24. Brown L F, Dvorak A M, Dvorak H. F. Leaky vessels, fibrin deposition, and fibrosis: a sequence of events common to solid tumors and to many other types of disease. Am Rev Respir Dis 1989; 140(4): 1104-7.

25. Weis S M, Cheresh D A. Pathophysiological consequences of VEGF-induced vascular permeability. Nature 2005; 437(7058): 497-504.

26. Kerbel R S, Kamen B A. The anti-angiogenic basis of metronomic chemotherapy. Nat Rev Cancer 2004; 4(6): 423-36.

27. Bertolini F, Paul S, Mancuso P, et al. Maximum tolerable dose and low-dose metronomic chemotherapy have opposite effects on the mobilization and viability of circulating endothelial progenitor cells. Cancer Res 2003; 63(15): 4342-6.

28. Browder T, Butterfield C E, Kraling B M, et al. Antiangiogenic scheduling of chemotherapy improves efficacy against experimental drug-resistant cancer. Cancer Res 2000; 60(7): 1878-86.

29. Shen D W, Fojo A, Chin J E, et al. Human multidrug-resistant cell lines: increased mdr1 expression can precede gene amplification. Science 1986; 232(4750): 643-5.

30. Wang K S, Kuhn R J, Strauss E G, Ou S, Strauss J H. High-affinity laminin receptor is a receptor for Sindbis virus in mammalian cells. J Virol 1992; 66(8): 4992-5001.

31. Ardini E, Pesole G, Tagliabue E, et al. The 67-kDa laminin receptor originated from a ribosomal protein that acquired a dual function during evolution. Mol Biol Evol 1998; 15(8): 1017-25.

32. Menard S, Tagliabue E, Colnaghi M I. The 67 kDa laminin receptor as a prognostic factor in human cancer. Breast Cancer Res Treat 1998: 52(1-3): 137-45.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1.-9. (canceled)

10. A method for treating a mammal suffering from a tumor which is resistant to an antitumor agent comprising metronomically administering to said mammal said antitumor agent and a Sindbis virus vector.

11. (canceled)

12. The method of claim 10, wherein said Sindbis virus vector is replication defective.

13. The method of claim 10, wherein said Sindbis virus vector comprises a Sindbis virus chimeric E2 envelope protein.

14. The method of claim 10, wherein said antitumor agent is Paclitaxel.

15.-16. (canceled)

17. A method for treating a mammal harboring a tumor comprising administering to a mammal in need of such treatment an amount effective to treat said tumor (a) CPT-11, and (b) a Sindbis virus vector, wherein the amounts of (a) and (b) in combination are effective to treat said tumor.

18. The method of claim 17 wherein said Sindbis virus vector is replication defective.

19. The method of claim 17 wherein said Sindbis virus vector is replication competent.

20. The method of claim 17 wherein said Sindbis virus vector comprises a chimeric E2 envelope protein.

21. The method of claim 17 wherein said Sindbis virus vector is replication competent.

22. (canceled)

23. The method of claim 17 wherein said Sindbis virus vector is replication defective.

24. The method of claim 17 wherein said Sindbis virus vector is replication competent.

25. The method of claim 17 wherein said Sindbis virus vector comprises a chimeric E2 envelope protein.

26.-29. (canceled)

30. A method of treating a malignant tumor in a mammal which comprises administering to a mammal in need of such treatment an amount of a Sindbis viral vector and an amount of an antitumor agent wherein the combined amount of said vector and said antitumor agent are effective to treat the tumor.
